(19) United States
(12) Patent Application Publication
(10) Pub. No.: US 2005/0216056 A1
(43) Pub. Date: Sep. 29, 2005

(54) Surgical dynamic compression staple

(75) Inventors: Antonio Valdevit, Fishkill, NY (US); Thomas Haher, New York, NY (US); Franco Coniglione, Farmington Hills, MI (US); Helmuth Kotschi, Middleburg Heights, OH (US)

Correspondence Address: HOFFMANN & BARON, LLP, 6900 JERICHO TURNPIKE, SYOSSET, NY, 11791, US

(73) Assignee: Lutheran Medical Center

(21) Appl. No.: 11/075,317

(22) Filed: Mar. 8, 2005

Related U.S. Application Data
(60) Provisional application No. 60/551,314, filed on Mar. 8, 2004.

(51) Int. Cl. A61B 17/64; A61B 17/08

(52) U.S. Cl. 606/219; 606/151

(57) ABSTRACT

A surgical staple generally including first and second arm members and an actuating component acting on the arm members. Each arm member has a jaw portion defining an interior jaw surface, a lever extension opposite the jaw portion and a hinge portion disposed between the jaw portion and the lever extension. The first arm member is pivotably attached to the second arm member about the hinge portion whereby the interior jaw surfaces of the first and second arm members face each other. The actuating component is disposed between the lever extensions of the first and second arm members for imparting a compressive force between the interior jaw surfaces of the first and second arm members.

### CROSS-REFERENCE TO RELATED APPLICATIONS

This application claims the benefit of U.S. Provisional Application No. 60/551,314, filed on Mar. 8, 2004.

### FIELD OF THE INVENTION

The present invention relates generally to surgical staples and clips and, more particularly, to a dynamic compression staple utilizing an actuating component for imparting a compressive force to the opposing jaws of the staple.

### BACKGROUND OF THE INVENTION

Medical mechanical fasteners have been used in various parts of the body, including spinal and gastrointestinal applications. Such devices are typically in the form of clamps, clips, staples, sutures, etc. which are able to apply sufficient constrictive forces to anatomical structures, such as blood vessels so as to limit or interrupt blood flow.

In order to avoid certain problems associated with conventional medical fasteners, the use of shape memory alloys (SMAs) has been proposed. For example, U.S. Pat. No. 4,485,816 discloses the use of a shape memory surgical staple for use in holding the edges of a wound together while it heals. Similarly, U.S. Pat. No. 5,022,563 discloses the use of shape memory sutures.

Shape memory alloys have the ability to “remember” specific shapes which correspond to particular metallurgical phases. If deformed, SMAs can be heated or cooled to invoke a phase transformation, which in turn, causes a transformation in shape. Shape memory alloys are characterized by a transition temperature or transition temperature range above which the predominant metallurgical phase is termed austenite and below which the predominant phase is termed martensite. The transformation temperatures of SMAs are commonly discussed with reference to Mₛ and M_{f}, the martensitic start and finish temperatures, respectively, and Aₛ and A_{f}, the austenitic start and finish temperatures, respectively. The transformation between these phases is reversible such that when alloys are deformed into some first configuration while in the austenitic state, cooled into a martensitic state, deformed into a second configuration, and then re-heated to the austenitic state, the alloy will revert back to the first configuration by virtue of the martensite-to-austenite phase transformation.

One of the problems, however, with SMA devices is that the change in temperature necessary to induce the required shape change can be procedurally difficult, and more importantly, can put the nearby tissue and surgical instrumentation at risk. In addition, it can be difficult to manufacture SMAs with the precise transformation temperatures necessary for surgical applications. It is therefore necessary to carefully monitor the temperature of such devices during shipping and storage so as to avoid phase transformations during this time.

Accordingly, it would be desirable to provide a medical fastener having a structure and geometry that minimizes the above drawbacks yet provides sufficient compression forces to anatomical structures.

### SUMMARY OF THE INVENTION

The present invention is a surgical staple generally including first and second arm members and an actuating component acting on the arm members. Each arm member has a jaw portion defining an interior jaw surface, a lever extension opposite the jaw portion and a hinge portion disposed between the jaw portion and the lever extension. The first arm member is pivotably attached to the second arm member about the hinge portion whereby the interior jaw surfaces of the first and second arm members face each other. The actuating component is disposed between the lever extensions of the first and second arm members for imparting a compressive force between the interior jaw surfaces of the first and second arm members.

In a preferred embodiment, the actuating component is a shape memory alloy, such as nickel titanium, which changes in size upon being subject to a temperature change. This change in size imparts the compressive force between the interior jaw surfaces of the first and second arm members. The shape memory alloy may be adapted to expand upon being subject to an increase in temperature, whereby the first and second arm members would be pivotably connected side-by-side so that an expansion of the shape memory alloy will cause a compressive force between the interior jaw surfaces. Alternatively, the shape memory alloy may be adapted to contract upon being subject to an increase in temperature, whereby the first and second arm members would be designed and assembled to cross over each other at the hinge portion so that a contraction of the shape memory alloy will cause a compressive force between the interior jaw surfaces.

In either case, at least one of the first and second arm member lever extensions preferably includes a slot for securing the actuating component thereto and the shape memory alloy is in the form of a block having a protrusion received in the lever extension slot. Also, the interior jaw surface is preferably formed with a serrated geometry.

The surgical staple of the present invention further preferably includes a pin connecting the hinge portions of the first and second arm members together and the hinge portion of the first and second arm members includes a cylindrical collet for retaining the pin therein. The pin may be formed integral with one of the first and second arm members. In any event, the pin preferably includes a retaining device, such as a rivet or a collapsible ball, for retaining the pin within the first and second arm members.

The preferred embodiments of the surgical dynamic compression staple as well as other objects, features and advantages of this invention, will be apparent from the following detailed description, which is to be read in conjunction with the accompanying drawing.

### BRIEF DESCRIPTION OF THE DRAWINGS

FIG. 1 is a top perspective view of the surgical dynamic compression staple formed in accordance with the present invention.

FIG. 2 is a top perspective view of the male component and pin of the surgical dynamic compression staple shown in FIG. 1.

FIG. 3 is a top perspective view of the female component of the surgical dynamic compression staple shown in FIG. 1.

FIG. 4 is a top perspective view of a preferred embodiment of the actuating component of the surgical dynamic compression staple shown in FIG. 1.

### DETAILED DESCRIPTION OF THE PREFERRED EMBODIMENTS

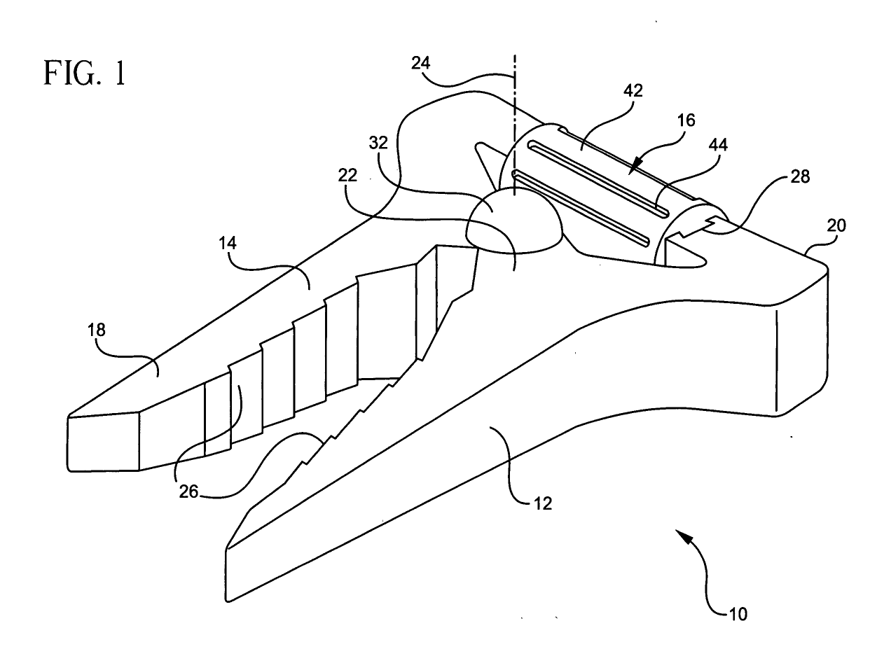

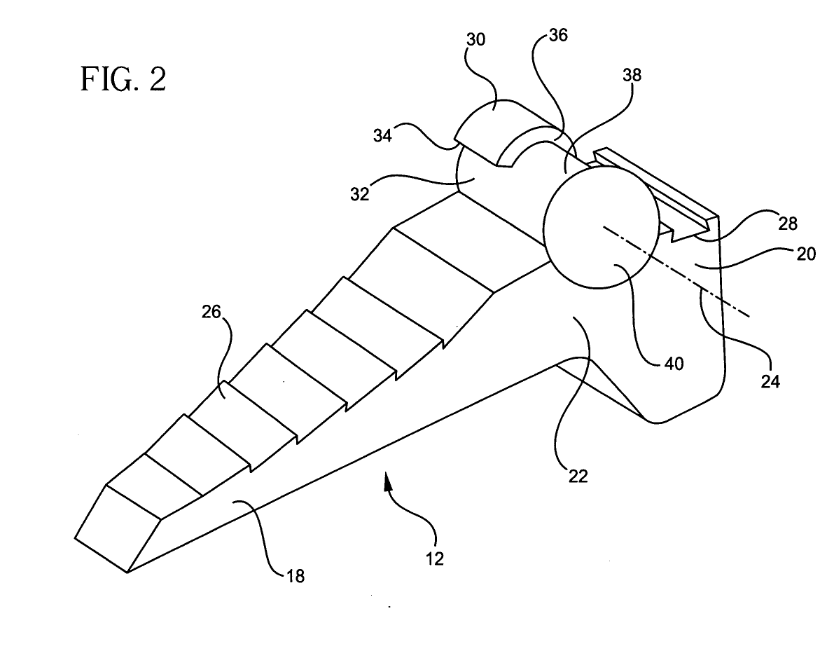

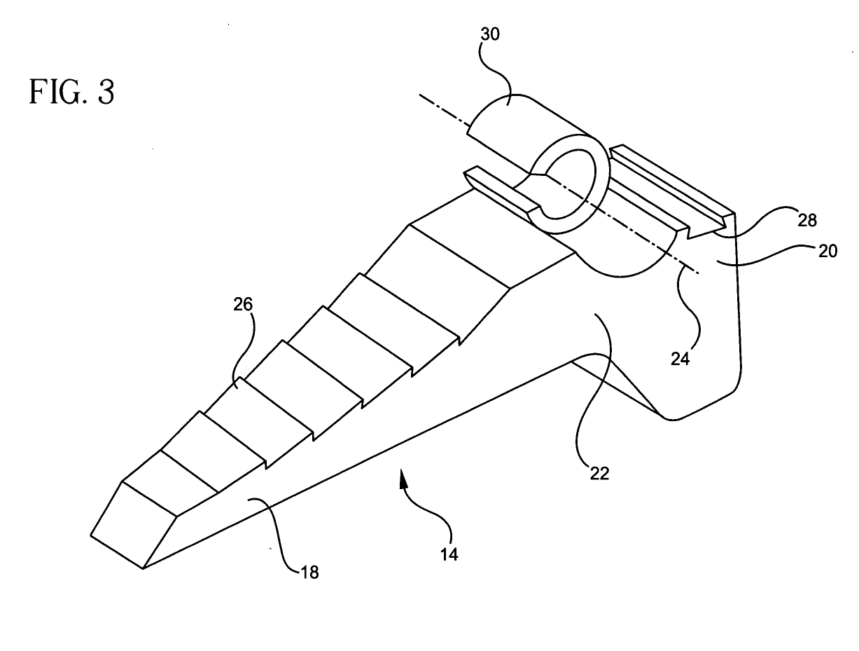

Referring to FIGS. 1-4, the surgical dynamic compression staple 10, according to the present invention, generally includes two similarly designed, pivotably connected, opposing arm members 12 and 14 and an actuating component 16 disposed between the arm members for imparting a compressive force between the arm members. Each arm member 12 and 14 generally includes a jaw portion 18, a lever extension 20 opposite the jaw portion and a hinge portion 22 disposed between the jaw portion and the lever extension. As will be discussed in further detail below, the hinge portion 22 defines a pivotal axis 24 between the arm members 12 and 14.

The arm members 12 and 14 may be fabricated from any rigid bio-compatible material suitable for the application of loads across the staple 10. In the preferred embodiment of the device, the first and second arm members 12 and 14 are fabricated from a conventional implant material, such as a titanium alloy and/or a stainless steel. In addition, both the internal and external profiles of the first and second arm members 12 and 14 possess a radius in order to minimize cavitation of the material to be stabilized upon staple compression. The profile of the compression arms 12 and 14 may be circular, triangular, rectangular or any other geometric shape. For example, the current embodiment shown in FIGS. 1-3 possesses a trapezoidal profile.

The jaw portion 18 of each arm member 12 and 14 preferably defines an interior jaw surface 26, which, when the staple 10 is assembled, faces the interior jaw surface of the opposing arm member. Preferably, the interior jaw surface 26 is formed with a serrated or knurled geometry for enhanced securing of the staple 10.

At a posterior end, opposite the jaw portion 18, each arm member 12 and 14 includes a lever extension 20 through which the actuating component 16 may be interfaced to impart a compressive force between the arm members. As shown in FIGS. 1-3, the lever extension 20 may include a slot or groove 28 for securing the actuating component 16 to the arms 12 and 14 of the staple 10. Of course, other cooperating engagement methods for securing the actuating member 16 between the arms 12 and 14 are contemplated by the present invention.

As mentioned above, the hinge portion 22 of the first and second arm members 12 and 14 defines a pivotal axis 24 between the jaw portion 18 and the lever extension 20. The hinge portion 22 preferably takes the form of a cylindrical collet 30 integrally formed on the arm member for receiving a pin 32. The collet 30 preferably includes a longitudinal opening 34 and an axial mechanical support surface stop 36. The pin 32 is retained within the collets 30 of the arm members 12 and 14 to assemble the arm members together. Upon assembly, however, the pin 32 is rotatably supported within at least one of the collets 30 to allow the first and second arm members 12 and 14 to pivot with respect to each other about the pivotal axis 24. Also, the mechanical support surface stop 36 of the first arm member collet 30 rests against the mechanical support surface stop of the second arm member collet so that the arm components 12 and 14 articulate in a circular fashion about the shaft of the pin 32.

The pin 32 retains the first arm member 12 and the second arm member 14 in pivoting relationship with respect to each other. The pin 32 may be fabricated separately as a single entity from a suitable bio-compatible implant material and inserted within the collets 30 of the arm members 12 and 14 upon assembly. In this manner, the pin 32 is rotatably seated in one or both collets 30.

Alternatively, the pin 32 may be formed as an integral component of or press-fit into a collet 30 of one of the arm members 12 or 14 prior to assembly. In this scenario, the arm member having the pin fixed thereto would be designated as the male arm member and the arm member having a collet for rotatably receiving the fixed pin would be designated the female arm member. Thus, the collet 30 of the male arm component 12 may be integrally formed with an exposed shaft portion 38 protruding from the collet in a direction perpendicular to a compression direction of the arm to form the pin 32.

In either embodiment, the pin 32 preferably includes an exposed shaft portion 38 terminating in a retaining device 40 that permits the second arm member 14 to rotatingly slide over the exposed shaft portion of the pin and become captured, thereby preventing disassembly of the device. In a preferred embodiment, the retaining device 40 is a rivet head, which can be crushed upon assembling the first and second arm members 12 and 14 to each other. However, the retaining device may alternatively take the form of a split or collapsible ball, a screw or other fastener.

As mentioned above, the actuating component 16 is disposed between the lever extensions 20 of the opposed arm members 12 and 14 and serves as a compression mechanism for the jaw portions 18 of the arm members. Depending on the design of the arm members 12 and 14, the actuating component 16 achieves this by supplying either an expansion or a contraction force between the lever extensions 20 located at the posterior end of the staple 10 opposite the pivotal axis 24. Such a force may be applied through a screw drive, a ratchet or any other means by which the two opposite surfaces of the arm lever extensions 20 may be displaced relative to each other, thereby causing the arms of the male and female components 12 and 14 to generate a compressive force acting along the interior jaw surfaces 26.

In a preferred embodiment, as shown in FIGS. 1-3, the arm members 12 and 14 are designed and assembled so that an expansion of the actuating member 16 will impart a compressive force between the opposing jaw portions 18 of the arms. Specifically, the arm members 12 and 14 shown in FIGS. 1-3 have jaw portions 18 and lever extensions 20 positioned side-by-side with respect to the pivotal axis 24 so that they will be disposed on the same longitudinal half of the staple 10 when assembled. Alternatively, the arm members 12 and 14 may be designed to cross over each other at the pivotal axis 24 in a scissors-type fashion. In this embodiment, a contraction of the actuating member 16 will be necessary to impart a compressive force between the opposing jaw portions 18 of the arms 12 and 14.

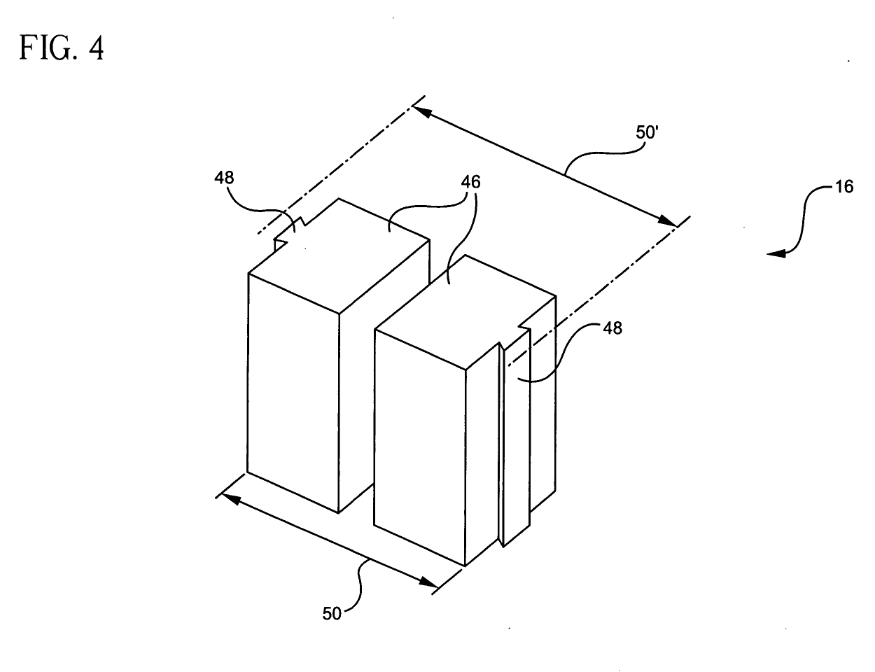

In the preferred embodiment, as shown in FIG. 1, the actuating component 16 takes the form of a single block of a biocompatible shape memory alloy disposed between the opposite surfaces of the arm lever extensions 20. More specifically, the actuating component 16 is preferably in the form of a cylindrical accordion 42 having a plurality of longitudinal slots 44 formed therein to facilitate expansion and contraction of the block. However, the actuating component may take any number of other alternative shapes or geometries, such as rectangular shapes or dumbbell shapes with spherical ends. Moreover, the actuating component 16 may take the form of two or more separate blocks 46, as shown in FIG. 4. For example, a block 46 of a preselected size shape memory alloy can be secured to the interior surface of the first arm extension 20 and another block is secured to the interior surface of the second arm extension. Each block 46 may include a protrusion 48 which is retained within the groove 28 of the lever extension 20.

Preferable biocompatible shape memory alloys include alloys of nickel titanium or NITINOL (an acronym for NIckel TItanium Naval Ordnance Laboratory, which is a tradename for a family of intermetallic materials containing a nearly equal mixture of nickel (55 wt. %) and titanium). Other elements can be added to adjust or “tune” the material properties. These alloys exhibit a first lateral dimension 50, as shown in FIG. 4, at a temperature below 98.6 degrees Fahrenheit (the normal human body temperature). However, upon implanting the staple 10 into a human body, the increase in temperature causes the lateral dimension 50 of the alloy block 42 or blocks 46 to increase to a second lateral dimension 50′. As the actuating component 16 thus expands, pressure is exerted on the interior surfaces of the arm extensions 20 causing the extensions to move further apart from each other. Due to the pivoting arrangement of the arms 12 and 14 about the pin 32, the expansion of the actuating component 16 results in a compressive force being applied between the opposing inner jaw surfaces 26 of the staple 10. This compressive force, in conjunction with the serrated geometry of the jaw surfaces, is used to anchor the staple 10 to an anatomical structure. If it is ever desired to remove the staple 10, the staple can be cooled, wherein the shape memory alloy actuating component 16 will return to its first contracted dimension thereby relieving the compressive force applied across the inner jaw surfaces.

Although the preferred embodiments of the present invention have been described with reference to the accompanying drawings, it is to be understood that the invention is not limited to those precise embodiments, and that other changes and modifications may be made by one skilled in the art without departing from the scope or spirit of the invention.

1. A surgical staple comprising:
first and second opposing arm members, each arm member having a jaw portion defining an interior jaw surface, a lever extension opposite said jaw portion and a hinge portion disposed between said jaw portion and said lever extension, said first arm member being pivotably attached to said second arm member about said hinge portion whereby said interior jaw surfaces of said first and second arm members face each other; and
an actuating component disposed between said lever extensions of said first and second arm members for imparting a compressive force between said interior jaw surfaces of said first and second arm members.

2. A surgical staple as defined in claim 1, wherein said actuating component comprises a shape memory alloy, said shape memory alloy changing in size upon being subject to a temperature change for imparting said compressive force between said interior jaw surfaces of said first and second arm members.

3. A surgical staple as defined in claim 2, wherein said shape memory alloy comprises nickel titanium.

4. A surgical staple as defined in claim 2, wherein said first and second arm members are pivotably connected side-by-side so that an expansion of said shape memory alloy will cause a compressive force between said interior jaw surfaces.

5. A surgical staple as defined in claim 4, wherein said shape memory alloy is adapted to expand upon being subject to an increase in temperature.

6. A surgical staple as defined in claim 2, wherein said first and second arm members cross over each other at said hinge portion so that a contraction of said shape memory alloy will cause a compressive force between said interior jaw surfaces.

7. A surgical staple as defined in claim 6, wherein said shape memory alloy is adapted to contract upon being subject to an increase in temperature.

8. A surgical staple as defined in claim 1, further comprising a pin connecting said hinge portions of said first and second arm members.

9. A surgical staple as defined in claim 8, wherein said hinge portion of said first and second arm members comprises a cylindrical collet for retaining said pin therein.

10. A surgical staple as defined in claim 8, wherein said pin is integral with one of said first and second arm members.

11. A surgical staple as defined in claim 8, wherein said pin includes a retaining device for retaining said pin within said first and second arm members.

12. A surgical staple as defined in claim 11, wherein said retaining device comprises a collapsible ball disposed at an end of said pin.

13. A surgical staple as defined in claim 1, wherein said interior jaw surface is formed with a serrated geometry.

14. A surgical staple as defined in claim 1, wherein at least one of said first and second arm member lever extensions includes a slot for securing said actuating component thereto.

15. A surgical staple as defined in claim 14, wherein said actuating component comprises a block of a shape memory alloy, said block having a protrusion received in said lever extension slot.
